SEQUENCE LISTING (14 sequences)

SEQ ID NO: 1 - DNA, 20 bp, Escherichia coli
  source 1..20: mol_type = unassigned DNA
aacacgcacggtgttacatt

SEQ ID NO: 2 - DNA, 20 bp, Escherichia coli
  source 1..20: mol_type = unassigned DNA
cggatcttccacaacacgca

SEQ ID NO: 3 - DNA, 20 bp, Escherichia coli
  source 1..20: mol_type = unassigned DNA
ttatcaccttggctgcaggc

SEQ ID NO: 4 - DNA, 20 bp, Escherichia coli
  source 1..20: mol_type = unassigned DNA
atgtaacaccgtgcgtgttg

SEQ ID NO: 5 - DNA, 20 bp, Escherichia coli
  source 1..20: mol_type = unassigned DNA
cgtgcgtgttgtggaagatc

SEQ ID NO: 6 - DNA, 20 bp, Escherichia coli
  source 1..20: mol_type = unassigned DNA
gaagatccggcctgcagcca

SEQ ID NO: 7 - DNA, 20 bp, Escherichia coli
  source 1..20: mol_type = unassigned DNA
atctctgtctatcgaagaat

SEQ ID NO: 8 - DNA, 91 bp, synthetic construct
  misc_feature 1..91: note = Synthetic Construct
  source 1..91: mol_type = other DNA
gttttagagctagaaatagcaagttaaaataaggctagtccgttatcaacttgaaaaagtgcgcacatgaggatcacccatgtgctttttt

SEQ ID NO: 9 - protein, 231 aa, synthetic construct
  REGION 1..231: note = Synthetic Construct
MGPASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSMSHQKIIQDLIAWIDEHIDQPLNIDVVAKKSGYSKWYLQRMFRTVTHQTLGDYIRQRRLLLAAVELRTTERPIFDIAMDLGYVSQQTFSRVFARQFDRTPSDYRHRL

SEQ ID NO: 10 - protein, 1368 aa, synthetic construct
  REGION 1..1368: note = Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - DNA, 1179 bp, synthetic construct
  misc_feature 1..1179: note = Synthetic Construct
  source 1..1179: mol_type = other DNA
aagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaactgtcgactcgagcctctatggattatcaccttggctgcaggccggatcttccacaacacgcacggtgttacattaggcataccggtcttgacagctagctcagtcctagggattgtgctagcgaattcattaaagaggagaaaggtaccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 12 - DNA, 2007 bp, synthetic construct
  misc_feature 1..2007: note = Synthetic Construct
  source 1..2007: mol_type = other DNA
aagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaactgtcgactcgagcctctatggattatcaccttggctgcaggccggatcttccacaacacgcacggtgttacattaggcataccggtcttgacagctagctcagtcctagggattgtgctagcgaattcattaaagaggagaaaggtaccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtggcggaggtagcatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 13 - protein, 1053 aa, synthetic construct
  REGION 1..1053: note = Synthetic Construct
AKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 14 - RNA, 99 nt, synthetic construct
  source 1..99: mol_type = other RNA
gggcacgggcagcttgccgggttttagagctgaaatagcaagttaaaacaaggctagtccgttatcaacttgaaaaagtggcaccgagtcggtgctttt

(19) United States
(12) Patent Application Publication
(10) Pub. No.: US 2025/0215420 A1
(43) Pub. Date: Jul. 3, 2025

(54) MULTIPLEXABLE CRISPR EDITORS UTILIZING INTRACELLULAR EVOLVED APTAMERS FOR ENDOGENOUS EFFECTOR RECRUITMENT

(71) Applicant: The Trustees of Boston College, Chestnut Hill, MA (US)

(72) Inventors: Jia NIU, Lexington, MA (US); Qiwen SU, Pawtucket, RI (US)

(73) Assignee: The Trustees of Boston College, Chestnut Hill, MA (US)

(21) Appl. No.: 19/054,596

(22) Filed: Feb. 14, 2025

Related U.S. Application Data
(63) Continuation of application No. 17/028,139, filed on Sep. 22, 2020, now abandoned.
(60) Provisional application No. 62/904,758, filed on Sep. 24, 2019.

(51) Int. Cl. C12N 15/10; C12N 9/22

(52) U.S. Cl. CPC C12N 15/1048; C12N 9/22; C12N 15/1068; C12N 2310/16; C12N 2310/20

(57) ABSTRACT

In one aspect, the disclosure relates to multiplexable, non-nuclease CRISPR editors comprising RNA aptamer sequences configured to bind to endogenous effector molecules. The disclosed CRISPR editors are small in size and can be delivered by a single adeno-associated virus capsid. Also disclosed are a method for intracellular evolution of aptamers, a method for introducing a genomic modifying event to a host cell using the disclosed CRISPR editors, and a host cell modified by the disclosed methods. This abstract is intended as a scanning tool for purposes of searching in the particular art and is not intended to be limiting of the present disclosure.

### CROSS REFERENCE TO RELATED APPLICATIONS

This application is a continuation of U.S. application Ser. No. 17/028,139, filed on Sep. 22, 2020, which claims the benefit of U.S. Provisional Application No. 62/904,758, filed on Sep. 24, 2019, the content of each of which is incorporated herein by reference in its entirety.

### STATEMENT REGARDING FEDERALLY SPONSORED RESEARCH OR DEVELOPMENT

This invention was made with U.S. Government support under grant number HG011027 awarded by the National Institutes of Health. The U.S. government has certain rights in the invention.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The Sequence Listing, in written computer readable format (CRF), is incorporated by reference in its entirety.

### FIELD OF THE INVENTION

The present disclosure is directed to the development of efficient CRISPR editors using intracellularly evolved aptamers for endogenous effector recruitment.

### BACKGROUND OF THE INVENTION

Complex and dynamic regulation of the expression of multiple genes is essential in almost all cellular functions. Therefore, the ability to precisely manipulate gene expression is crucial to understand the complex functions of gene network and engineer them for tailored properties. However, until recently, simple, robust, and general tools for genomic and epigenomic editing have remained elusive. While RNA interference (RNAi) technology allows for sequence-specific gene silencing in eukaryotic cells, its efficiency and specificity require further improvement to be seen as viable options for genome editing. Furthermore, although RNAi can be used for gene silencing, this type of editing is transient and cannot uncover regulatory mechanisms involved at the DNA and epigenetic levels. Beyond RNAi, customizable DNA-binding proteins such as zinc-finger proteins and transcription activator-like effectors (TALEs) have been associated with nucleases (e.g., restriction endonuclease FokI) for sequence-specific DNA cleavage. Despite their high genomic editing activities, zinc finger proteins and TALEs require large protein complexes for specific gene targeting, making their construction and delivery challenging, as well.

RNA-based riboswitches, ribozymes, and affinity reagents (herein termed “aptamers”) have been employed in nature to regulate gene expression. Most existing synthetic aptamers and riboswitches are generated through a process called systematic evolution of ligands by exponential enrichment (SELEX), in which a random RNA library is enriched by affinity to the solid support-bound target. A previous study demonstrated a strategy for directly evolving functional RNA motifs intracellularly to regulate transcription in a yeast three-hybrid system, laying the foundation for the development of synthetic RNA-based regulators of gene expression in mammalian cells. Nevertheless, the proper function of existing examples of RNA-based regulators is still dependent on the specific sequence context. Multiplexable, “plug-and-play” types of RNA regulators remain elusive.

The discovery of CRISPR-Cas RNA-guided DNA endonuclease heralded a new era for genomic and epigenomic editing. Following the simple Watson-Crick base pairing rules, the Cas9-sgRNA complex can target specific DNA loci in a programmable manner. The only requirement of a DNA sequence targeted by CRISPR (the “protospacer”) is that it must have a “protospacer-adjacent motif” (PAM). Thanks to its simplicity in targeting mechanism and robust targeting efficiency, the genomic editing functionality of the CRISPR system has gone beyond DNA cleavage—covalent conjugation or non-covalent recruitment of effector proteins to the Cas9 protein or the sgRNA have enabled their co-localization with the Cas9-sgRNA complex to specific DNA loci for gene expression regulation. Compared to CRISPR endonucleases, the non-nuclease CRISPR editors are attractive alternatives in therapeutic applications, as they avoid causing double-strand breaks, a known source of cytotoxicity associated with CRISPR nuclease-based genomic editing.

Sequence-specific genome editing enabled by CRISPR-Cas technology has revolutionized biomedical sciences and has significant therapeutic potentials for a wide range of diseases. The highly modular and programmable nature of the CRISPR system has provided significant opportunities for investigation into the orchestrated interactions of genetic elements within complex gene networks through multiplexed genomic editing. Beyond the original CRISPR RNA-guided endonuclease, non-nuclease CRISPR editors have recently attracted increasing attention, but the utility of these systems has been hampered by two major challenges: 1) a limited ability to multiplex distinct functions at multiple sites and 2) inefficient intracellular delivery. The first challenge is primarily caused by the limited scope of existing orthogonal and/or inducible mediators that can link locus-specific recognition by CRISPR with the recruitment of effectors for genomic editing. While adeno-associated virus (AAV) is a promising delivery vector to address the second challenge, the size of existing non-nuclease CRISPR editors exceeds the capacity of a single AAV capsid, resulting in a significant reduction of the delivery efficiency (FIGS. 1A, 2).

With non-nuclease CRISPR editors, a “dead” Cas9 protein (dCas9) with a mutation to deactivate the nuclease activity is usually employed to avoid undesired DNA cleavage (see Ref. 58). One study demonstrated that fusing a transcriptional activator VP64 domain covalently to dCas9 enabled robust transcriptional activation. Other work suggests that engineering the sgRNA to incorporate known RNA aptamers recognizing specific tags (e.g., phage coat proteins MCP, PP7, etc.) enables the recruitment of the effector proteins (e.g., VP16) fused to these tags. It has also been found that the sgRNA can accommodate large insertions in several stem-loop regions without losing the locus-targeting function or the ability to complex with Cas9 protein.

The excellent locus-targeting capability of the CRISPR system has also enabled locus-specific recruitment of epigenetic modulators and nucleobase-editing proteins. DNA methyltransferase DNMT3A and ten-eleven translocation methylcytosine dioxygenase 1 (Tet1) have been tethered with dCas9 to allow DNA methylation and demethylation at the respective targeted loci. Likewise, histone-modifying enzymes, such as acetyltransferase p300 and histone demethylase LSD1, were fused to dCas9 for locus-specific histone modification. One exciting advancement in CRISPR-mediated genomic editing is the nucleobase editors, which tethering a cytidine deaminase domain to Cas9 allows for a highly specific C to U (equivalent to T) conversion within a small window at the sgRNA binding site. Compared to CRISPR endonucleases, the nucleobase editor can achieve editing at the single-base level in high precision and avoid the formation of insertions and deletions caused by nonhomologous end-joining after CRISPR endonuclease-induced double-strand break. As such, this technology has promising therapeutic prospects in treating various diseases caused by single-base mutations in the genome.

AAV-based gene delivery vector is an attractive solution to the challenges associated with the intracellular delivery of the CRISPR system. AAV can infect both dividing and non-dividing cells, does not integrate its DNA into the host genome, and has relatively low immunogenicity. In addition, multiple serotypes of AAV that target different tissues can be adapted to gene delivery, providing the opportunity for tissue-specific delivery of CRISPR editors. Nevertheless, the packaging capacity of ˜4.5 kb significantly limits the ability of a single AAV vector to deliver the entire CRISPR system (the encoding gene of SpCas9, the commonly used version of Cas9, is 4.2 kb) (FIG. 2). Current solutions to this challenge include delivery using multiple AAV vectors, split Cas9, smaller Cas9 variants, and transgenic Cas9-expressing animals. Although these solutions are effective in laboratory, their low delivery efficiency and the requirement of transgenesis still pose a major challenge for AAV-mediated delivery of non-nuclease CRISPR editors in therapeutic applications.

The modularity and programmability of the CRISPR system have enabled multiplexed targeting of different gene loci simultaneously, an ability that is critical for the investigation into the interplay of different elements in a complex gene network. Various sgRNA libraries have been designed to allow genome-wide gene targeting, and the targeted genes can be edited by the editing domain associated with the Cas9-sgRNA complex. Accordingly, an array of genome-wide manipulations using CRISPR genomic editors have been reported, including gene knockout, transcriptional activation, transcriptional repression, locus-specific imaging, and epigenetic modification. A previous study has demonstrated that modular RNA scaffolds fused to sgRNA can be used to recruit different effectors fused with a tag that allows simultaneous activation and repression at different gene loci. The multiplexability of this system is enabled by the combination of multiple pairs of RNA aptamers and their cognate proteins used as tags fused to the effectors. A similar approach called CRISPR-Display uses functional RNA motifs fused to sgRNA to execute multiplexable functions spanning fluorescent imaging and effector recruitment. However, the limited scope of existing RNA aptamer-tag pairs has imposed significant constraint over the scale of multiplexing, in addition to the required delivery of the exogenous effectors fused with the tags.

In summary, non-nuclease CRISPR editors provide promising opportunities for multiplexable regulation of the complex gene network, but existing systems are limited by the scarce RNA aptamer-tag pairs and the large size exceeding the capacity of a single AAV vector. The development of compact and multiplexable non-nuclease CRISPR editors can significantly improve our ability to understand and engineer the complex functions of the gene network. These needs and other needs are satisfied by the present disclosure.

### SUMMARY OF THE INVENTION

In accordance with the purpose(s) of the present disclosure, as embodied and broadly described herein, the disclosure, in one aspect, relates to a CRISPR editor comprising a miniaturized, catalytically-inactive Cas9 protein fused to an RNA sequence, wherein the RNA sequence comprises a guide sequence capable of hybridizing to a target sequence and wherein the RNA sequence further comprises an aptamer. In some aspects, the CRISPR editor further comprises a functional protein such as, for example, a protein that inhibits DNA repair, fused to the modified Cas9 protein.

In another aspect, the guide sequence of the CRISPR editor is configured to hybridize to a DNA sequence within from 50 to 1000 bases of a transcription start site for a gene, a gene to be repressed, a site for methylation or demethylation, a site for RNA or DNA cleavage, a site for modification of a nucleotide residue, or a combination thereof.

In still another aspect, the aptamer is configured to bind to an endogenous effector such as, for example, an RNA polymerase enzyme or subunit, a transcriptional activator or repressor, an epigenetic regulator, an O-GlcNAc transferase, an O-GlcNAcase, an E3 ubiquitin ligase, or a DNA repair enzyme.

In one aspect, the CRISPR editor can be packaged into a single adeno-associated virus (AAV) capsid such as a capsid of serotype AAV2, AAV-DJ, or another serotype. In any of these aspects, the CRISPR induces low immunogenicity in a host organism.

In another aspect, disclosed herein is a multiplexing CRISPR editor comprising at least two individual CRISPR editors as disclosed herein, wherein the two individual CRISPR editors have different RNA sequences. In some aspects, the guide sequences of the individual CRISPR editors are different and in other aspects, the aptamers are configured to bind to different endogenous effectors.

In some aspects, the disclosure relates to a method for introducing a genomic modifying event or a protein modification to at least one host cell, the method comprising contacting the host cell with at least one disclosed CRISPR editor. Also disclosed are host cells modified by the method.

In one aspect, disclosed herein is a method for intracellular selection of aptamers for a target molecule, the method comprising: providing a randomized RNA sequence library, inserting the RNA sequence library into one or more RNA guide sequences configured to hybridize to a target sequence; constructing non-nuclease CRISPR editors where the guide sequences are fused to reporter constructs and modified Cas9 proteins; transforming host cells with the CRISPR editors containing randomized RNA sequences; culturing the host cells in the presence of a target molecule; visualizing the host cells, wherein the host cells that include a unique RNA sequence that binds the target molecule activate the reporter construct, providing a signal; and sorting the host cells such that cells providing the signal are separated from host cells that do not provide the signal. In some aspects, the randomized RNA sequence library can be pre-enriched using SELEX. In another aspect, the selection can be repeated one, two, three, or more times for more robust intracellular enrichment.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1B show a comparison between a previous multiplexable non-nuclease CRISPR editor (FIG. 1A) and a multiplexable non-nuclease CRISPR editor according to the present disclosure (FIG. 1B). Challenges with previous editors include: a) multiplexability limited by few known aptamer-tag pairs; b) systems are generally too large for AAV delivery; and c) the exogenous effectors can be immunogenic (FIG. 1A). Solutions provided in the present disclosure include: a) the ability to evolve new aptamers intracellularly; b) the disclosed methods target endogenous effectors that do not require delivery; c) the disclosed editors consist of miniaturized dCas9 (SEQ ID NO. 10) and sgRNA-aptamer chimera, small in size and suitable for single AAV delivery; and d) the disclosed editors are less immunogenic and use non-immunogenic endogenous effectors (FIG. 1B).

FIG. 2 shows a comparison between existing CRISPR editors based on aptamers for effector recruitment (left) and a CRISPR editor disclosed herein (right).

FIG. 3A shows sgRNA structure an insertion site of the disclosed aptamer library. FIG. 3B shows the design of intracellular aptamer selection constructs in E. coli cells.

FIG. 4 shows linear maps of three plasmids used for aptamer-mediated CRISPR activation in E. coli. The selection plasmid (SP) pQS1.3 encodes the gRNA scaffold, which includes the gRNA J1.3 and MS2 aptamer. The gRNA sequence J1.3 guides the catalytically dead Cas9 protein (dCas9, SEQ ID NO. 10), encoded in accessory plasmid (AP) pQS7, to the synthetic promoter upstream of the Kan-GFP reporter gene in reporter plasmid (RP) pQS50 (SEQ ID NOS. 11-12).

FIGS. 5A-5C show activation of gene expression with different CRISPRa target sites. FIG. 5A: the J1 synthetic promoter has six potential gRNA target sites (J1.1 to J1.6) on the template and non-template strands upstream of a weak BBa_J23117 promoter driving a GFP reporter gene. Target sites for J1.1 to J1.6 are available as SEQ ID NOs. 1-6, with SEQ ID NO. 7 as an off-target (control) site. Target sites can be on the non-template DNA strand (i.e., SEQ ID NOs. 1-3) or the template DNA strand (i.e., SEQ ID NOs. 4-6). FIG. 5B: a 56-fold increase in GFP expression is observed from the gRNA target J1.3, located 100 bases upstream of transcriptional start site (TSS), while a 22-fold increase in GFP expression is observed with the gRNA target J1.6. Background fluorescence level was measured in the parental MG1655 E. coli strain containing the GFP reporter gene (MG1655+GFP). GFP levels were measured in late stationary phase. FIG. 5C: gene activation requires all components of CRISPRa. Values reported are GFP fluorescence levels measured by flow cytometer. Values are median+standard deviation for at least 3 biological replicates.

FIGS. 6A-6B show GFP expression of CRISPRa with MCP mutants. FIG. 6A: all the MCP mutants retained some CRISPRa function. FIG. 6B: gene activation from CRISPRa with MCPT45L is comparable to background fluorescence. Values reported are GFP fluorescence levels measured by flow cytometer. Values are median+standard deviation for at least 3 biological replicates.

FIG. 7 shows measurements of binding affinity of aptamer to MCP by enzyme-linked oligonucleotide assay (ELONA). Microtiter plates were coated with 10 μg/mL recombinant MCP-SoxS_{R93A}-his₆ and a concentration series of 0.25-2,500 nM of 5′-biotinylated aptamer. K_{D}=26.31±6.76 nM. Absorbance was measured at 652 nm using a plate reader. Values are median±standard deviation for at least 3 biological replicates.

FIG. 8 shows LC-MS analysis of recombinant MCP-SoxS_{R93A}-his₆ with an expected molecular weight of 26,708 Da.

FIG. 9 shows LC-MS analysis of recombinant MCP_{T45L}-SoxS_{R93A}-his₆ with an expected molecular weight of 26,720 Da.

FIG. 10 shows a general scheme for intracellular selection of aptamers.

FIGS. 11A-11B show model screening with library and consensus MS2 aptamer against wild-type MCP. FIG. 11A: model screening results. Gene compositions before and after antibiotic selection were compare following AleI digestion, revealing strong enrichment for the consensus aptamer. FIG. 11B: fluorescence measurements of cells recovered in model screening. Negative control: library aptamer with MCP. Positive control: MS2 aptamer with MCP. Values reported are GFP fluorescence levels measured by flow cytometer. Values are median±standard deviation for at least three biological replicates. See also SEQ ID NOs. 11-12 for constructs useful herein.

FIGS. 12A-12C show FACS screening of pre-enriched aptamers from antibiotic selection. FIG. 12A: general scheme for the FACS screen. FIG. 12B: fluorescent cells within the specified gate (black rectangle) were collected. FIG. 12C: gene composition before and after sorting was compared with AleI digestion.

FIGS. 13A-13B show intracellular aptamer selection in mammalian cells. FIG. 13A: three plasmids (SP, AP, and RP) are designed to deliver sgRNA-aptamer library chimera, dSaCas9 and genes necessary for adeno-associated virus serotype 2 (AAV2) amplification, and the reporter construct, respectively. FIG. 13B: genetic elements of SP and AP. ITR is the packaging signal sequence.

FIG. 14A shows a reporter construct for c-Myc aptamer selection. FIG. 14B shows a reporter construct for Tet1 aptamer selection. The DazI is a germ cell-specific promoter and will be hypermethylated in the HEK293T cell line. The demethylation of Snrpn gene by Tet1 can turn on the expression of eGFP. FIG. 14C shows a reporter construct for cytidine deaminase aptamer selection. A UGI is added to dSaCas9. When the inactive Cre with a Y324H mutation is repaired by cytidine deaminase, it can excise the RFP gene flanked by the IoxP sites and express functional eGFP.

FIG. 15 shows a schematic of a genome-scale multiplexed screen combining transcriptional activation and epigenetic editing.

FIG. 16 shows a schematic of a genome-scale kinase substrate screen based on CRISPR-mediated base editing.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE INVENTION

Disclosed herein is a new class of non-nuclease CRISPR editors that incorporate evolvable RNA aptamer motifs in the sgRNA to enable recruitment of various effectors in both bacterial and mammalian cells. Further disclosed is an RNA library that has been developed through an intracellular functional selection to identify aptamers that can locus-specifically recruit endogenous effectors to induce the expression of a reporter gene. In one aspect, the effector-recruiting aptamers have been integrated with sgRNA in a chimeric RNA construct, which is further combined with a miniaturized Cas9 gene to constitute the complete CRISPR editor gene for AAV-mediated delivery. In another aspect, the disclosed CRISPR editors can be applied to genome-scale, multiplexed screens and execute transcriptional, epigenetic, and post-translational editing programs to reveal the interplay of the genetic elements that confer drug resistance in cancer cells.

### Non-Nuclease CRISPR Editors

In one aspect, disclosed herein are CRISPR editors that include a modified Cas9 protein (e.g., dCas9, SEQ ID NO. 10) fused to an RNA sequence, wherein the RNA sequence includes a guide sequence capable of hybridizing to a target sequence and further includes an aptamer. In one aspect, the modified Cas9 protein is miniaturized and/or is catalytically-inactive.

In some aspects, the CRISPR editors also include a functional protein fused to the modified Cas9 protein. In another aspect, the functional protein can be a protein that inhibits DNA repair. In some aspects, the CRISPR editors can modify nucleic acids by a method such as, for example, cytosine deamination. In a further aspect, deamination of cytosine leads to the presence of uracil in DNA and certain DNA repair enzymes such as, for example, uracil DNA glycosylase inhibitor, repairs uracil bases in DNA through a base-excision mechanism. In a still further aspect, if cytosine removal by cytosine deamination through use of a CRISPR editor is intentional, inhibition of DNA repair can ensure that the mutation persists in the DNA. In other aspects, other functional proteins can be fused to the modified Cas9 protein.

In one aspect, the guide sequence is configured to hybridize to a DNA sequence within from about 50 to about 1000 bases of a gene to be transcribed (e.g., a transcription start site), a gene to be repressed, a site for methylation or demethylation, a site for RNA or DNA cleavage, a site for modification of a nucleotide residue (e.g., deamination), or a combination thereof. In one aspect, the guide sequence hybridizes to a DNA sequence about 50, 100, 150, 200, 250, 300, 350, 400, 450, 500, 550, 600, 650, 700, 750, 800, 850, 900, 950, or about 1000 bases from the genomic or epigenomic location where modification is desired, or a combination of any of the foregoing values, or a range encompassing any of the foregoing values. In another aspect, the guide sequence can hybridize to the transcribed strand of DNA or the complementary, non-transcribed strand of the DNA.

### Endogenous Effectors

In one aspect, the RNA aptamer is configured to bind to an endogenous effector. In a further aspect, the endogenous effector can be an RNA polymerase enzyme or subunit, a transcriptional activator, a transcriptional repressor, an epigenetic regulator, or a DNA repair enzyme or subunit. In aspects where the aptamer binds to an enzyme subunit, other subunits of the enzyme can recruit and bind to the complex of aptamer and subunit to form a complete, catalytically-active enzyme. In a further aspect, recruitment of endogenous effectors allows for the CRISPR editors to be smaller in size, which can facilitate packaging into a viral vector for delivery to cells.

In another aspect, the RNA aptamer is configured to bind to an O-GlcNAc transferase or another enzyme capable of glycosylating a protein. In one aspect, an O-GlcNAc transferase catalyzes the addition of an N-acetylglucosamine to a hydroxyl-containing amino acid residue in a protein. In one aspect, the RNA aptamer is configured to bind to an O-GlcNAcase or another enzyme capable of removing one or more glycosyl groups from a protein. Further in this aspect, the O-GlcNAcase catalyzes the removal of an N-acetylglucosamine residue from a serine or threonine in a protein. In either of these aspects, glycosylation or deglycosylation (i.e., removal or addition of a sugar or amino sugar) of a protein can change protein stability and/or regulate protein activity.

In still another aspect, the RNA aptamer is configured to bind an E3 ubiquitin ligase. In one aspect, an E3 ubiquitin ligase is a protein that recruits an E2 ubiquitin-conjugating enzyme that has been loaded with ubiquitin and transfers that ubiquitin to a target protein substrate. In a further aspect, tagging a protein substrate with ubiquitin targets that protein substrate for destruction.

### Adeno-Associated Virus Delivery

In one aspect, the CRISPR editors disclosed herein can be packaged in a single adeno-associated virus (AAV) capsid. In another aspect, packaging in a single AAV capsid is more efficient for delivery of the CRISPR editors than other delivery methods such as, for example, delivery of two separate AAV capsids containing different components to the cells and more survivable than transformation by methods such as, for example, electroporation. In one aspect, the AAV capsid can be of any serotype or pseudotype of AAV including, but not limited to, AAV2 or AAV-DJ.

In any of these aspects, the CRISPR editor induces low immunogenicity in a host organism.

### Multiplexed Genome Editing

In one aspect, the ability to perform multiplexed editing of the genome as disclosed herein provides significant opportunity to investigate the interplay of various elements in the complex gene network. Since its advent, the CRISPR-Cas system has demonstrated unique advantages over previous genomic editing tools such as sequence specificity, programmability, and the ease of implementation. However, in some aspects, the utility of the existing non-nuclease CRISPR editors is still hampered by major challenges including limited ability of multiplexing and inefficient intracellular delivery. Thus, in another aspect, the present disclosure provides a new class of compact, multiplexable CRISPR editors that allow the genomic editing at transcriptional, epigenetic, and post-translational levels. In a further aspect, at the center of these CRISPR editors are intracellularly evolved RNA aptamers that are capable of recruiting endogenous effectors independently of additional labeling and tagging, thereby enabling the locus-specific co-localization of the CRISPR editors and the endogenous effectors. In one aspect, the present disclosure provides novel CRISPR-based constructs to enable the intracellular selection of aptamers in both bacterial and mammalian cells for robust and highly specific recognition of various effectors regulating distinct cellular functions. In still another aspect, the disclosed methods and constructs can be further implemented into the multiplexable interrogation and manipulation of multiple distinct cellular functions including transcriptional activation, epigenetic remodeling, and DNA nucleobase editing. In one aspect, compared to existing CRISPR editors, the new CRISPR editors described herein do not require the delivery of exogenous effectors, allowing for the encoding gene to be efficiently delivered by a single adeno-associated virus (AAV) vector. In one aspect, these CRIPSR editors can be used to execute multiplexed editing programs in genome-scale screens to identify novel genetic elements responsible for the emergence of drug resistance in cancer. In one aspect, the tools described herein significantly improve researchers' ability to understand and engineer the gene network in various applications spanning fundamental research and therapeutics.

In one aspect, disclosed herein is a multiplexing CRISPR editor composed of at least a first CRISPR editor as disclosed herein and a second CRISPR editor as disclosed herein. In one aspect, the first CRISPR editor has a first RNA sequence and the second CRISPR editor has a second RNA sequence, and the RNA sequences are different from one another.

In some aspects, the first RNA sequence includes a first guide sequence and the second RNA sequence includes a second guide sequence and the first and second guide sequences are configured to hybridize to different target DNA sequences. In other aspects, the first RNA sequence includes a first aptamer and the second RNA sequence includes a second aptamer, and the first and second aptamers are configured to bind to different endogenous effectors.

In any of these aspects, multiplexing CRISPR systems containing three, four, five, or more different CRISPR editors should also be considered disclosed.

### Genomic Modifying Events

In another aspect, disclosed herein is a method for introducing a genomic modifying event to at least one host cell, the method including contacting the host cell with at least one CRISPR editor as disclosed herein. In another aspect, the genomic modifying event can include activation of transcription, repression of transcription, methylation or demethylation of DNA, RNA or DNA cleavage, deamination of a nucleotide residue, or a combination thereof. Also disclosed are host cells modified by the disclosed methods. In one aspect, the host cell can be a mammalian cell, a non-mammalian vertebrate cell (e.g., bird, reptile, amphibian, fish), an invertebrate cell (e.g., mollusk, arthropod, or the like), a plant cell, a bacterial cell, a protozoal cell, fungal cell, or an archaeal cell. In any of these aspects, when a multiplexing CRISPR editor is used to contact the host cells, two or more genomic modifying events can occur (e.g., demethylation of DNA and deamination of a cytosine base, or activation of transcription of one gene and repression of transcription of another gene, and the like).

### Intracellular Evolution of RNA Aptamers

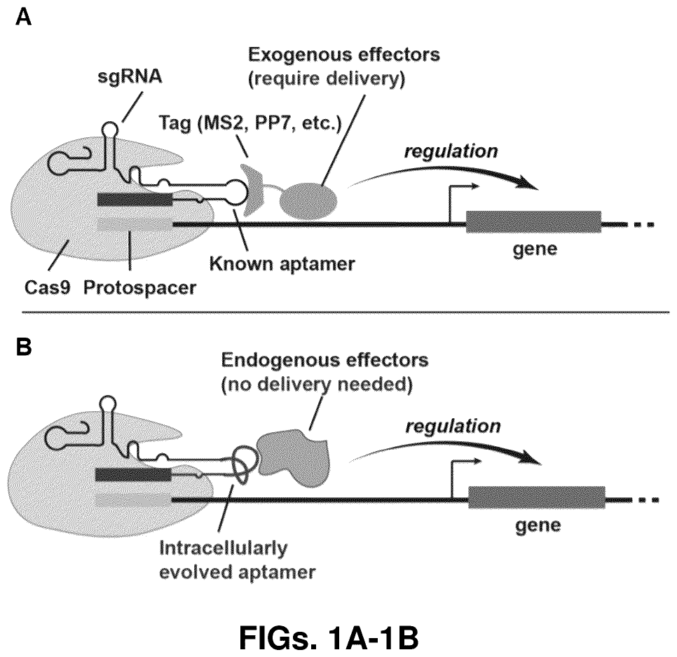

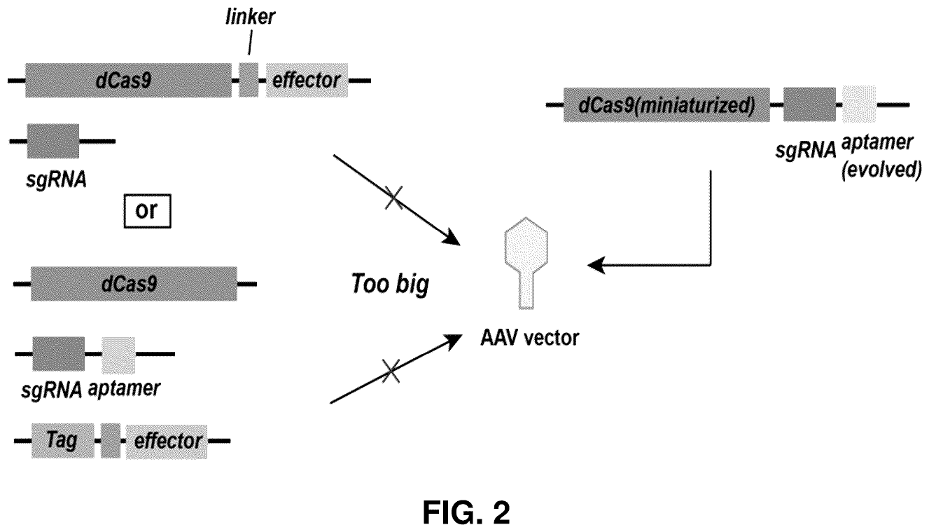

In one aspect, disclosed herein is a new class of CRISPR editors that incorporate intracellularly evolved RNA aptamer motifs into the short guide RNA (sgRNA) to enable specific recognition and recruitment of effectors in both bacterial and mammalian cells (FIGS. 1A-1B). In a further aspect, these effectors include bacterial RNA polymerase, viral transcriptional activator VP16, transcriptional activator c-Myc, epigenetic regulator Tet1, and DNA repair enzyme cytidine deaminase. In one aspect, the disclosed methods enable the intracellular selection of the effector-targeting aptamers in both bacterial and mammalian cells. In a further aspect, compared to the RNA aptamers integrated into the existing CRISPR systems, the aptamers disclosed herein are directly evolved in the intracellular environment where the CRISPR editors operate, thereby avoiding loss of affinity and potential off-target effects that are commonly associated with the in vitro selected aptamers when applied in vivo. In a further aspect, the newly-evolved aptamers are fundamentally distinct from the existing paradigm and are designed to directly target endogenous effectors, allowing the resultant CRISPR editors to be significantly more compact and modular for effector recruitment. In one aspect, the present disclosure enables efficient packaging of the CRISPR editors into single AAV capsids and their intracellular delivery. In a further aspect, this technology can be applied to understanding the complex interplay between genes and drugs in the emergence of drug resistance in cancer cells and, ultimately, in identifying the “hidden” mechanisms that cancer cells exploit to evade the inhibition of signaling pathways. In one aspect, towards these goals, genome-scale, the present disclosure demonstrates the ability to be used in multiplexed screens of genetic elements edited at the transcriptional, epigenetic, and post-translational levels by the CRISPR editors to probe the genetic cues for the emergence of drug resistance in cancer. In one aspect, the disclosed editors and methods have greatly expanded the toolkits for studying cancer drug resistance, thus creating new therapeutic opportunities. In a still further aspect, the present technology is also readily applicable to other biomedical research areas impacting human health that involve genetic factors.

In one aspect, disclosed herein is a method of intracellular selection of aptamers for a target molecule, the method including the following steps:
a. providing a randomized RNA sequence library;
b. inserting the RNA sequence library into a plurality of guide RNA sequences, wherein the plurality of guide RNA sequences are configured to hybridize to a target sequence;
c. constructing a plurality of non-nuclease CRISPR editors, wherein the plurality of guide RNA sequences are fused to a reporter construct and a modified Cas9 protein;
d. transforming a population of host cells with the plurality of non-nuclease CRISPR editors, such that each host cell includes no more than one unique RNA sequence from the randomized RNA sequence library;
e. culturing the host cells in the presence of a target molecule;
f. visualizing the host cells, wherein the host cells that include a unique RNA sequence that binds the target molecule activate the reporter construct, providing a signal; and
g. sorting the host cells such that cells that provide the signal from activating the reporter construct are retained;
   wherein a unique RNA sequence that binds the target molecule is an aptamer for the target molecule.

In a further aspect, the method can optionally include pre-enriching the randomized RNA sequence library with sequences that bind to a target molecule using SELEX prior to performing step (b). In a still further aspect, intracellular selection can be carried out multiple times. Stated differently, steps (e), (f), and (g) can be repeated one, two, three, or more times following performance of the method in order to further evolve or enrich the aptamer sequences.

In one aspect, the reporter construct can include a gene that expresses a fluorescent protein (e.g., a gene for EGFP), a gene that binds to a dye molecule, a gene that confers resistance to an antibiotic, or a combination thereof.

In still another aspect, once the aptamers disclosed herein are evolved intracellularly, the CRISPR editors are not required to continue to activate the reporter construct and this element can then be eliminated from the working system. In one aspect, interaction of the aptamers and target molecules can be studied by any means known in the art including, but not limited to, UV/vis or fluorescence spectroscopy, circular dichroism spectropolarimetry, nuclear magnetic resonance spectroscopy, surface plasmon resonance, another method, or a combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a short guide RNA,” “an effector,” or “an aptamer,” includes, but is not limited to, mixtures or combinations of two or more such short guide RNAs, effectors, or aptamers, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “epigenome editing” or “epigenomic editing” refers to modification of the epigenome (i.e., structure DNA and histone proteins as well as chemical compounds and proteins that can attach thereto) at specific sites using molecules targeted to those sites. Examples of epigenome editing include, but are not limited to, methylation and demethylation, activation or repression of transcription through binding of a molecule to a gene or promoter, and the like. In one aspect, the CRISPR editors disclosed herein are useful for epigenomic editing.

As used herein, “genome editing” or “genomic editing” refers to modification of a DNA (including non-coding DNA) or gene sequence in an organism through an insertion, deletion, modification, replacement, or the like. In some aspects, the CRISPR editors disclosed herein are useful for genomic editing. Examples of genome editing include, but are not limited to, replacement of a single base with another base such as, for example, deamination of cytosine to form uracil, which is an analog of thymine.

“Aptamers” as used herein are small oligonucleotide sequences that bind to a specific target molecule. In one aspect, aptamers can be constructed from RNA. In another aspect, the RNA of an aptamer may adopt a particular secondary and/or tertiary structure useful for forming contacts with the target molecule. In some aspects, aptamer binders for a target molecule can be selected from a large random sequence pool.

“Systematic evolution of ligands by exponential enrichment” or “SELEX” as used herein is an in vitro selection method for enriching a random pool of oligonucleotides with sequences that bind to a target molecule. In a typical SELEX procedure, a random pool of oligonucleotide sequences, typically having primers at the 5′ and 3′ ends, are exposed to a target ligand and those that do not bind the target are removed. In a further aspect, multiple rounds of selection can be performed to further narrow the pool of oligonucleotides (i.e., aptamers). In one aspect, the pool of oligonucleotide sequences useful herein as aptamers can be pre-enriched by a SELEX procedure prior to intracellular evolution of the final aptamers.

As used herein, “intracellular evolution” of aptamers refers to a method of selecting aptamers in host cells, wherein aptamers that bind the target molecule (here, an endogenous effector) cause expression of a reporter gene. In one aspect, aptamers pre-enriched by SELEX can be incorporated into the guide RNAs of the CRISPR editors disclosed herein, such that the CRISPR editors bind to a target DNA sequence and present the aptamers to the intracellular environment, whereby the aptamers can bind the target molecule. In a further aspect, the target molecule can be an endogenous effector such as, for example, bacterial ω-subunit of E. coli RNA polymerase, wherein binding of the target molecule recruits other subunits of the E. coli RNA polymerase, thereby transcribing a target gene such as, for example, a reporter gene. In one aspect, during intracellular evolution of aptamers, the CRISPR editors are packaged and delivered such that only one aptamer sequence is present per cell. Thus, cells not having aptamers with high binding affinities to the target effectors will not effectively express the reporter gene or will not do so above background levels, and cells having the constructs with the highest affinities can be selected by an appropriate method (e.g., by fluorescence activated cell sorting for eGFP as a reporter gene) and subjected to subsequent rounds of intracellular evolution of aptamers.

A “riboswitch” is a segment of RNA that binds a small molecule, leading to a change in production of a protein produced by the RNA. In some aspects, the aptamers disclosed herein can be or contain riboswitches. Further in these aspects, when a target molecule (e.g., a fluorescent dye or an antibiotic) binds the riboswitch, activity of the disclosed CRISPR editors can be altered.

“CRISPR” is an acronym for “clustered regularly interspaced short palindromic repeats” and refers to a family of DNA sequences found in prokaryotic genomes. CRISPR sequences are derived from viruses (i.e., bacteriophages) that previously infected the prokaryotes in which they are found. In a still further aspect, CRISPR sequences are used by prokaryotes to detect and destroy DNA from similar viruses during subsequent infections. Meanwhile, “Cas” enzymes (short for “CRISPR-associated”) use CRISPR sequences to recognize and cleave bacteriophage DNA that is complementary to the CRISPR sequences. In some aspects, the “CRISPR-Cas” system can be used for genomic editing in any organism by contacting cells from that organism with at least one CRISPR-Cas construct that acts by cleaving the genome at a desired location, allowing for the removal of existing genes or the addition of new genes. “Cas9” or “CRISPR associated protein 9” is an RNA-guided DNA endonuclease enzyme that can induce site-directed double-strand breaks in DNA.

In some aspects, the CRISPR editors disclosed herein include a “miniaturized” version of the Cas9 protein, wherein the size of the folded protein has been reduced by truncation or other removal of at least some amino acids. In one aspect, miniaturizing the Cas9 protein allows for packaging into a single adeno-associated virus (AAV) capsid for more efficient delivery into cells. In another aspect, the miniaturized Cas9 version may be “catalytically-inactive,” lacking some or all of its native endonuclease function.

In one aspect, disclosed herein are “non-nuclease CRISPR editors.” In a further aspect, the non-nuclease CRISPR editors as used herein include a miniaturized and/or otherwise catalytically-inactive Cas enzyme (i.e., a Cas enzyme lacking nuclease activity) that can assist a guide nucleotide sequence in binding to DNA. In a still further aspect, the non-nuclease CRISPR editors disclosed herein further contain an aptamer that can recruit and bind to an effector such as, for example, a transcription activator or repressor, a protein or peptide that modifies a nucleobase (e.g., cytidine deaminase enzyme), a protein or peptide that methylates or demethylates a nucleobase, thereby affecting gene expression, or the like.

As used herein, “genomic modifying event” refers to a change made in the genome or epigenome of an organism through use of the non-nuclease CRISPR editors disclosed herein. In one aspect, the genomic modifying event can be or include activating transcription of a gene, repressing transcription of a gene, demethylation of DNA, methylation of DNA, RNA cleavage, DNA cleavage, deaminating one or more nucleotide residues, another event, and/or a combination thereof

In one aspect, as disclosed herein, an “effector” is a small molecule, protein, peptide, or the like, that selectively binds to a protein and regulates its activity. In a further aspect, the aptamers disclosed herein can recruit effectors such as, for example, RNA polymerases, transcriptional activators, transcriptional repressors, epigenetic regulators, DNA repair enzymes, and the like. An “endogenous” effector is one naturally present in a host cell (e.g., in E. coli, a subunit of an E. coli RNA polymerase). In one aspect, the non-nuclease CRISPR editors disclosed herein include aptamer sequences useful for binding endogenous effectors in order to perform an action at or near a target site in the host cell genome.

A “guide RNA,” “gRNA,” “short guide RNA,” or “sgRNA” as used herein refers to a short, non-coding RNA sequence that binds to a complementary target DNA sequence. In one aspect, the non-nuclease CRISPR editors disclosed herein include sgRNA sequences that have been modified to include aptamer sequences as discussed herein. In a further aspect, the sgRNA sequences bind at or near target sites in host cell DNA such that the endogenous effectors recruited by the aptamer sequences can perform functions such as, for example, transcription activation or repression, genome or epigenome editing, and the like.

An “adeno-associated virus” or AAV is a small virus that infects humans and/or other primates. AAV infect both dividing and non-dividing cells, typically do not cause diseases, and provoke only a mild immune response, thus making them useful as vectors for gene therapy applications. In one aspect, AAV capsids are small and thus unsuitable for containing large CRISPR constructs in a single capsid. In a further aspect, however, delivery using two or more AAV capsids can be inefficient. In an alternative aspect, miniaturizing the Cas9 protein in the CRISPR editors disclosed herein enables packaging of the non-nuclease CRISPR editors in a single AAV capsid. In a further aspect, various AAV serotypes and/or pseudotypes can be used including, but not limited to, AAV1, AAV2, AAV4, AAV5, AAV6, AAV7, AAV8, AAV9, AAV-DJ, AAV2/5, AAV2/7, AAV2/8, or another AAV pseudotype, depending on the tissues and/or cell types in which genomic and/or epigenomic editing is desired. In one aspect, AAV2 is a useful AAV serotype for delivery of the non-nuclease CRISPR editors disclosed herein.

“Multiplexing” as used herein refers to the ability of the disclosed systems and methods to carry out distinct genomic modifying events at different sites or loci. In one aspect, previously known CRISPR editors were incapable of effectively multiplexing due to delivery issues (e.g., are too large for packaging in a single AAV capsid) and/or due to limited availability of aptamers that effectively pair with and/or recruit effectors having the desired functions. In one aspect, the non-nuclease CRISPR editors disclosed herein are capable of effective multiplexing, thus representing an improvement over known CRISPR editors.

An “enzyme linked oligonucleotide assay” or ELONA refers to an assay where an antibody is immobilized on a surface, the antibody binds to a ligand of interest, and a fluorescently-labeled oligonucleotide aptamer that also binds to the ligand of interest can be used as a reporter for target detection. In one aspect, ELONA can be used to determine the affinity of an aptamer for a particular target ligand (e.g., an effector for the non-nuclease CRISPR editors disclosed herein).

As used herein, “transcriptional editing” or “post-transcriptional modification” refers to alteration of an RNA primary transcript after transcription. In one aspect, the non-nuclease CRISPR editors disclosed herein can perform transcriptional editing by recognizing a target sequence in an intracellular RNA such as, for example, a tRNA or mRNA molecule or another RNA molecule and performing a function using a recruited endogenous effector that binds to the aptamer sequence of the non-nuclease CRISPR editor including, but not limited to, repression or activation of translation, nucleobase editing (e.g., cytosine deamination), or the like.

“Immunogenicity” as disclosed herein refers to the ability of a foreign or exogenous substance to provoke an immune response in the body of an animal including a human or another mammal, or another non-mammalian vertebrate. In one aspect, the non-nuclease CRISPR editors disclosed herein are “non-immunogenic” (i.e., they do not provoke an immune response) or exhibit “low immunogenicity,” that is, while an immune response is provoked, only a low level of anti-CRISPR-editor antibodies are generated. In another aspect, low immunogenicity is associated with few or no side effects in a subject treated with the non-nuclease CRISPR editors disclosed herein.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Insertion of RNA Aptamer Into CRISPR sgRNA

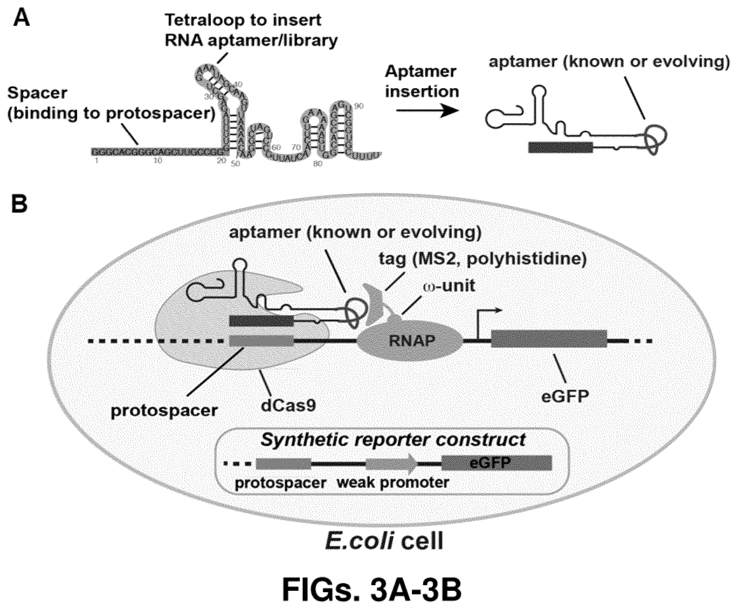

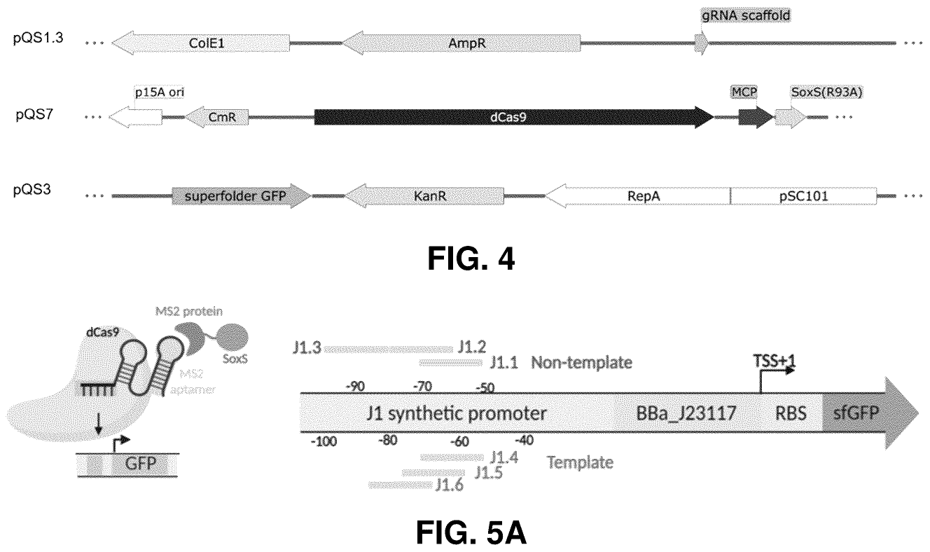

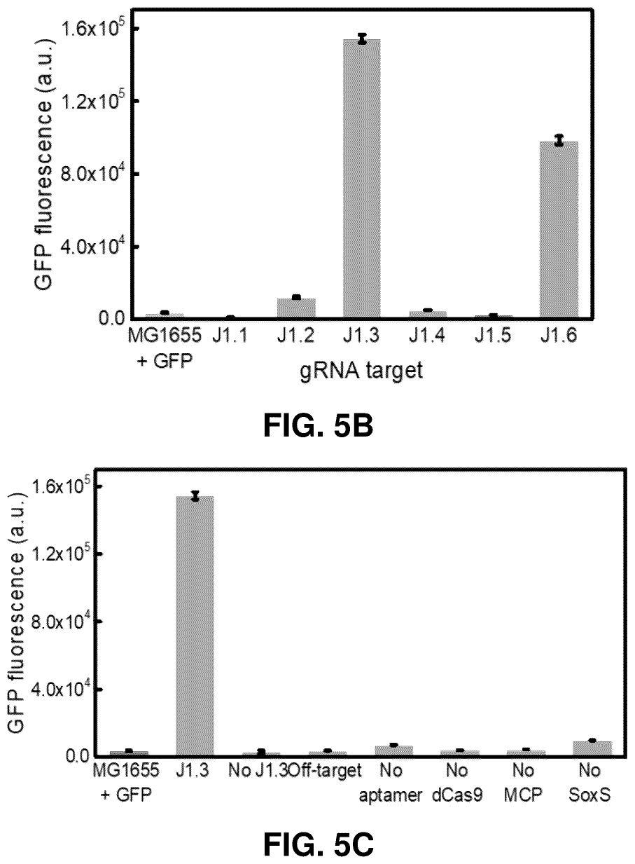

To test the ability of the CRISPR system to accommodate the insertion of an RNA aptamer motif into the sgRNA and allow for locus-specific effector recruitment in E. coli cells, a construct was first designed to insert a known RNA aptamer against bacteriophage MS2 coat protein (MCP) into the tetraloop region of sgRNA (FIG. 3A, SEQ ID NO. 8). Correspondingly, MCP was fused with the SoxS transcriptional activator. Three plasmids (SP, AP, and RP) were designed to introduce into the cell the sgRNA-aptamer chimera, the MCP-SoxS fusion along with dCas9 (SEQ ID NO. 10), and a synthetic reporter construct, respectively (FIG. 4). Following a design principle established by a previous study, the synthetic reporter was constructed to control the expression of enhanced green fluorescent protein (eGFP) by CRISPR co-localized with RNA polymerase through aptamer-MCP interaction (FIG. 3B). This synthetic reporter construct contained a weak promoter and multiple protospacer sequences to be targeted by sgRNA to control levels of gene activation (FIG. 5A).

The distance of the sgRNA target from the transcription start site was also tested by using this reporter. Data presented in FIGS. 5B-5C shows that significant GFP expression is observed from the CRISPR-aptamer conjugate (CRISPRa) with gRNA J1.3 (on-target), but GFP levels that are comparable to background were expressed when one of the CRISPRa components was removed, including gRNA-aptamer J1.3 (no J1.3), MS2 aptamer (no aptamer), catalytically-dead Cas9 (no dCas9), and MS2 coat protein (no MCP). Low GFP levels were also observed when an off-target gRNA was used that had no binding site on the J1 synthetic promoter. Reporter gene expression arose from the recruitment of the activator, MS2 coat protein (MCP) fused with transcriptional activator SoxSR93A, by the MS2 aptamer to the synthetic promoter (SEQ ID NO. 9).

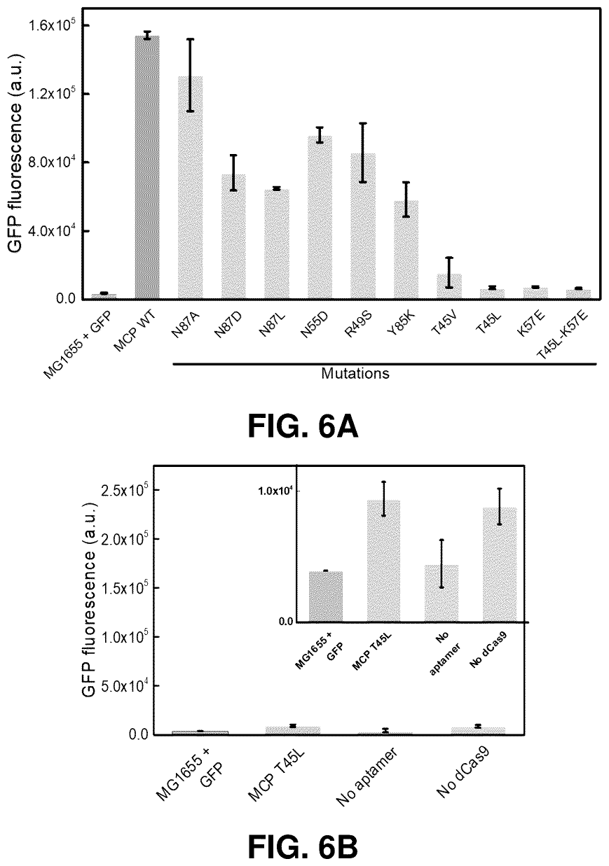

Data presented in FIG. 6A shows all the MCP mutants retain CRISPRa function. A point mutation at MCP T45L resulted in a 23-fold decrease in GFP expression compared to wild type MCP, while MCP Y85K, N87A, T45V, N87D, and R49S led to substantial decrease in GFP expression. Data presented in FIG. 6B demonstrates that when MS2 aptamer hairpin was removed from the CRISPRa with MCP T45L, levels of GFP expression were comparable to background fluorescence of the parental MG1655 E. coli strain with reporter gene (MG1655+GFP). When dCas9 was omitted, similar GFP activity to CRISPRa was observed.

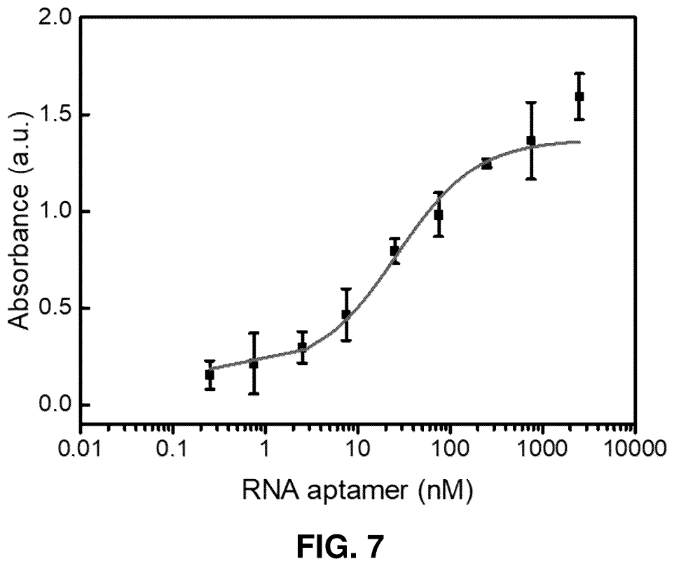

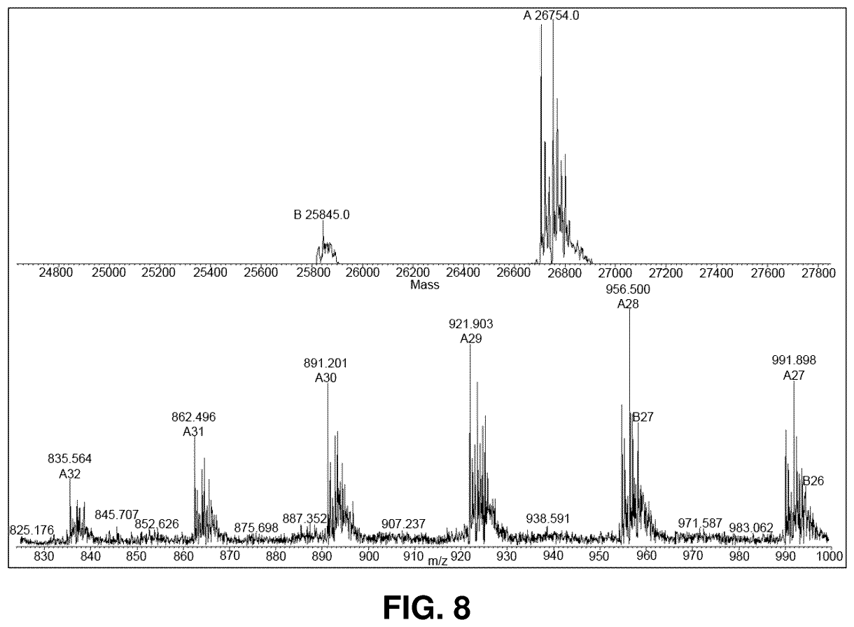

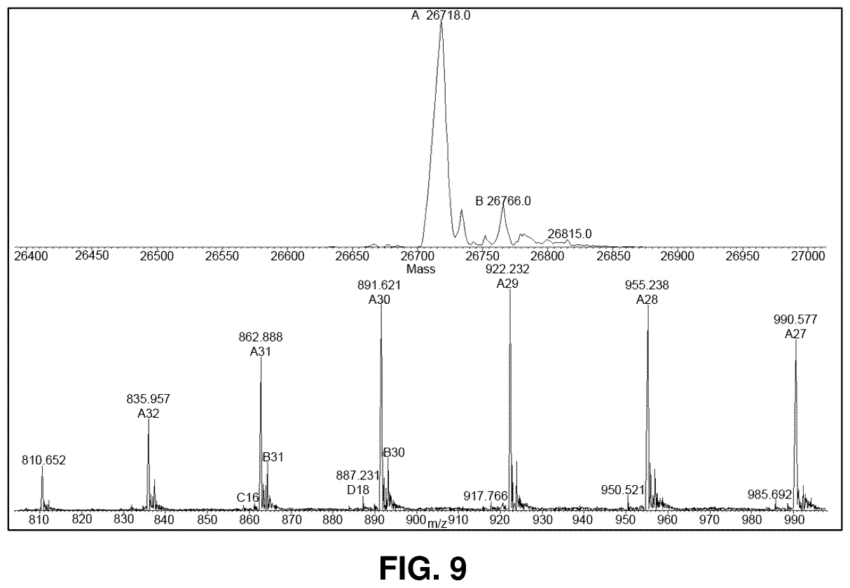

The MS2 aptamer was characterized for its affinity with recombinant MCP and mutants by using enzyme-linked oligonucleotide assay (ELONA) (FIG. 7). LC-MS characterizations of recombinant proteins are shown in FIGS. 8-9.

### Example 2: Aptamer Evolution and Selection

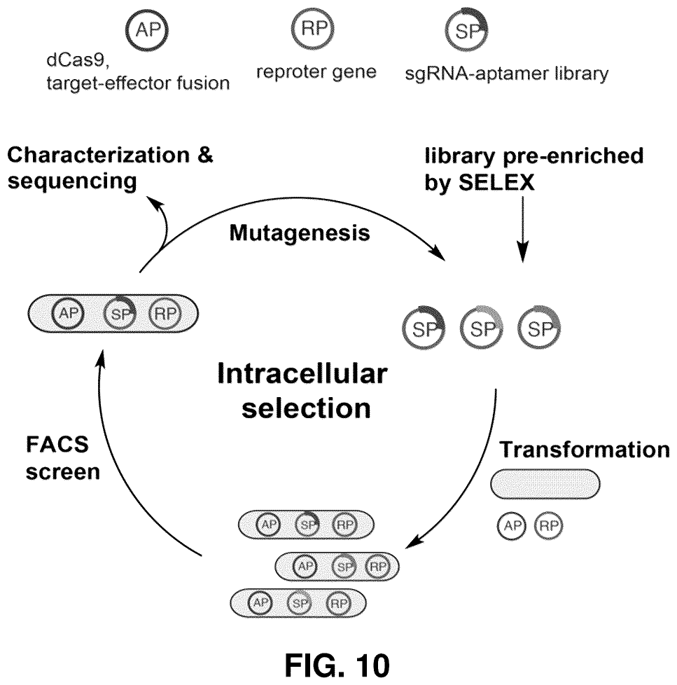

The disclosed methods employ a CRISPR-based construct for the intracellular selection of RNA aptamers in bacterial and mammalian cells. Disclosed herein are de novo aptamers that to MCP mutants through intracellular selection (FIG. 10).

To evolve the aptamer, an N40 randomized library (i.e., consisting of 40 consecutive random bases) was pre-enriched for MCP mutants binding through SELEX. This step is believed to have improved selection outcome as its throughput of 10¹²˜10¹⁴ far exceeded that of intracellular selection (10⁷˜10⁸) and provided better coverage of the N40 library (˜10²⁴). The pre-enriched library was inserted into the tetraloop of the sgRNA. Transformation of the chimera into E. coli was controlled such that each cell had only one unique sequence. The RNA aptamers that can bind to the MCP mutant target protein enable the co-localization of the RNA polymerase at the promoter region to activate expression of kanamycin resistance protein in fusion with eGFP. E. coli cells were then selected with increasing concentration of kanamycin and sorted by fluorescence-activated cell sorting (FACS).

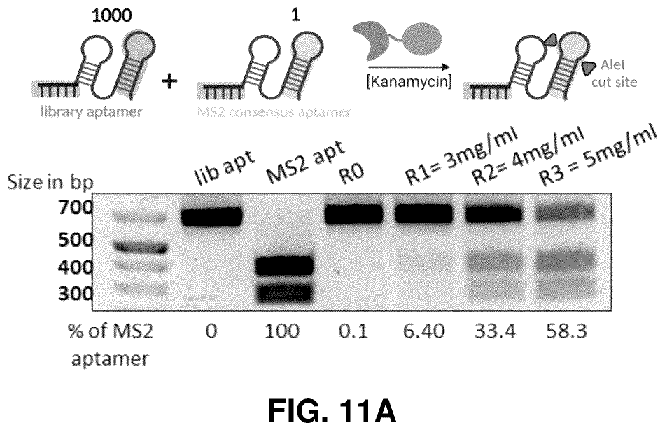

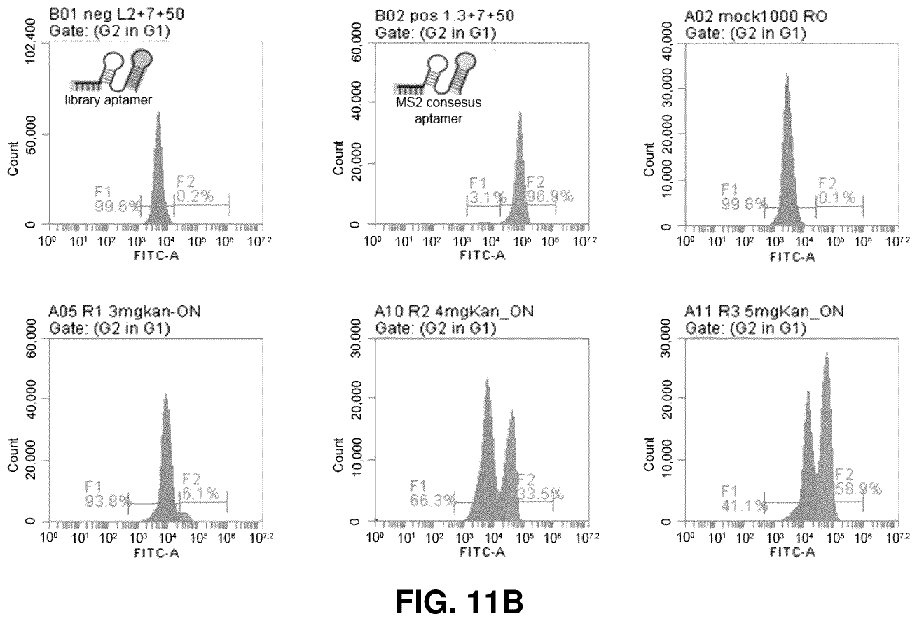

To validate the proposed intracellular aptamer selection, a model screening was completed with cell mixtures of 1000:1 of library: consensus MS2 aptamer against wild-type MCP. After three rounds of selection with kanamycin increments, a 583-fold enrichment of the consensus aptamer was observed by AleI restriction enzyme digestion and eGFP analyses (FIGS. 11A-11B). Active aptamer binders were strongly enriched from a mixture containing predominantly weak and inactive aptamers.

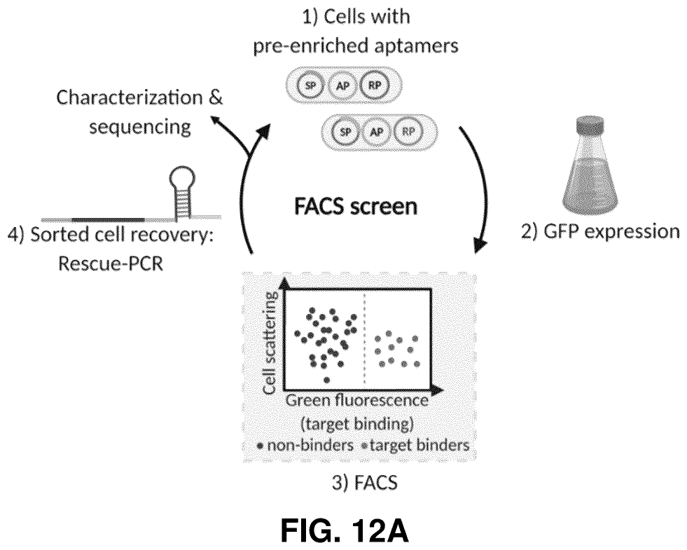

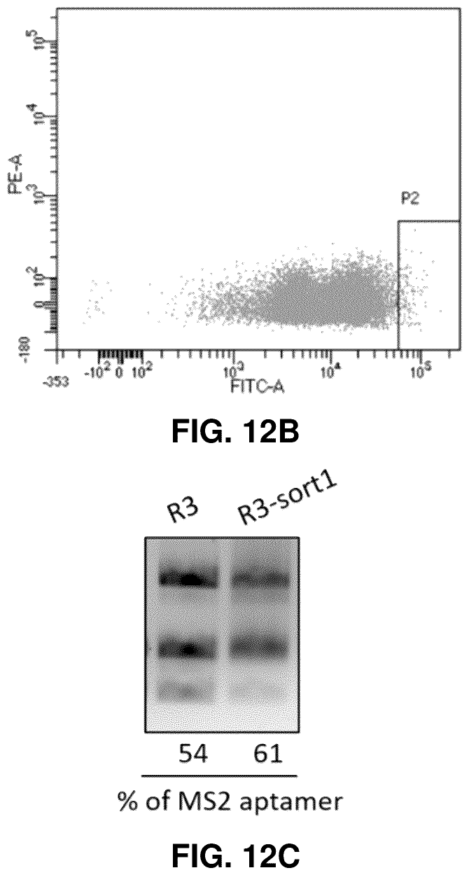

FIG. 12A is a schematic showing sorting of E. coli cells with enriched aptamer sequences by FACS, followed by characterization and sequencing. The consensus MS2 aptamer was further enriched by 1.2-fold with only one round of sorting (FIGS. 12B-12C). These results demonstrated that CRISPR-mediated intracellular aptamer selection was successful using a combination of antibiotic-fluorescent gene reporter and FACS.

The selected aptamers were characterized for their affinities via independent affinity characterizations including surface plasmon resonance (SPR) and isothermal titration calorimetry (ITC).

### Synthetic Riboswitches

The above-described intracellular aptamer selection strategy was extended to generate synthetic riboswitches to control the activity of the CRISPR editors using a small molecule ligand (e.g., tetramethylrosamine or tetracycline). The known aptamers of these small molecule ligands were fused with the starting RNA library via a randomized oligoribonucleotide linker. The fusion was first selected by positive selection for the cells expressing fluorescence in the presence of the ligand. The enriched population was also subjected to a negative selection in which the cells showing no fluorescence in the absence of the ligand were enriched.

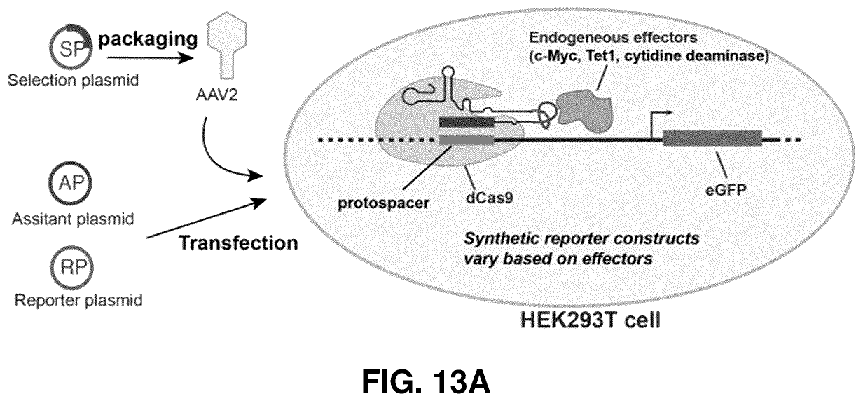

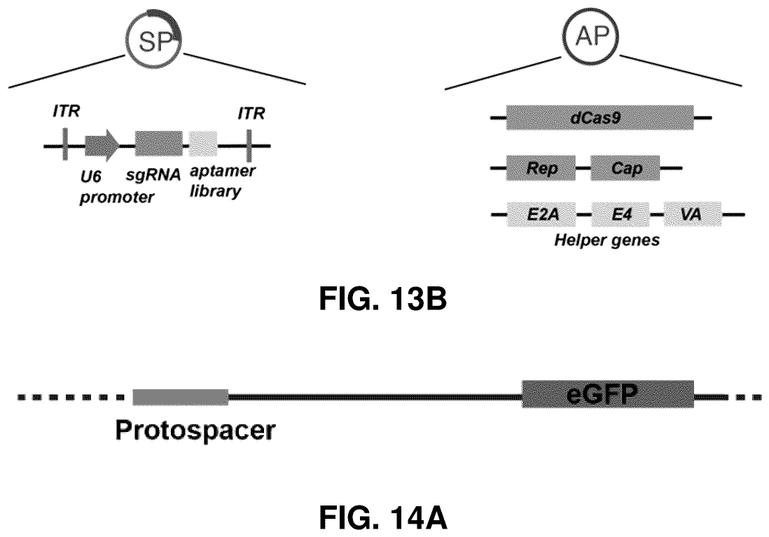

The intracellular aptamer selection system in mammalian cells also consists of three main components: the sgRNA-aptamer library chimera, the dCas9 protein (SEQ ID NO. 10), and the reporter construct (FIG. 13A). The selection construct design in mammalian cells was similar to the one in bacterial cells, in which an RNA library was inserted into the tetraloop of the sgRNA. The activity of effector proteins in gene expression regulation was reported by eGFP expression. The sgRNA-library chimera was complexed with dSaCas9, a miniaturized dCas9 protein (SEQ ID NO. 13) encoded by a 3.2 kb gene. A proof-of-principle aptamer selection targeting the VP16 transcriptional activator was performed to validate the selection system. RNA sequences that can bind VP16 and recruit it to the proximity of the protospacer by the CRISPR construct led the activation of eGFP expression and enrichment in the subsequent FACS sorting. The library was pre-enriched by SELEX before performing intracellular selection.

Key to the successful intracellular aptamer selection was the physical compartmentalization of individual library members, which amounted to the delivery of no more than a single library member per cell. However, unlike bacterial cells, mammalian cells typically do not replicate and amplify plasmids from transfection. Without amplification of the incoming plasmid, the signal from a single copy of the reporter was too weak to be detected by FACS. To overcome this challenge, plasmids allowing intracellular delivery of the sgRNA-aptamer library chimera by AAV2 vectors were designed (FIG. 13B). The AAV2 genome can replicate and amplify in the host cell with its Rep gene, Cap gene, and additional helper genes from adenovirus provided by plasmids in trans, allowing the generation of a large number of copies from a single delivered plasmid.

### Example 3: Recruitment of Effectors

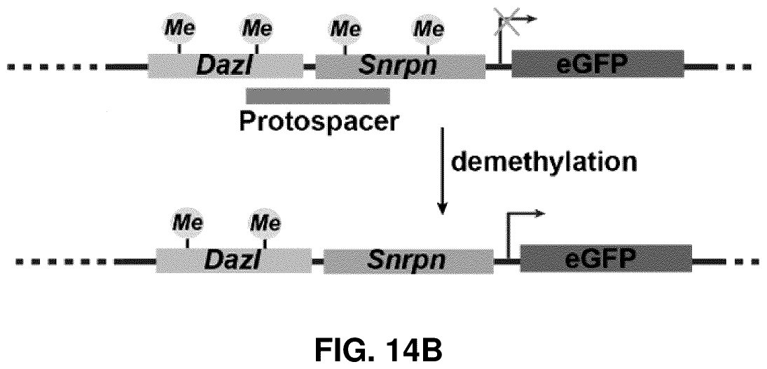

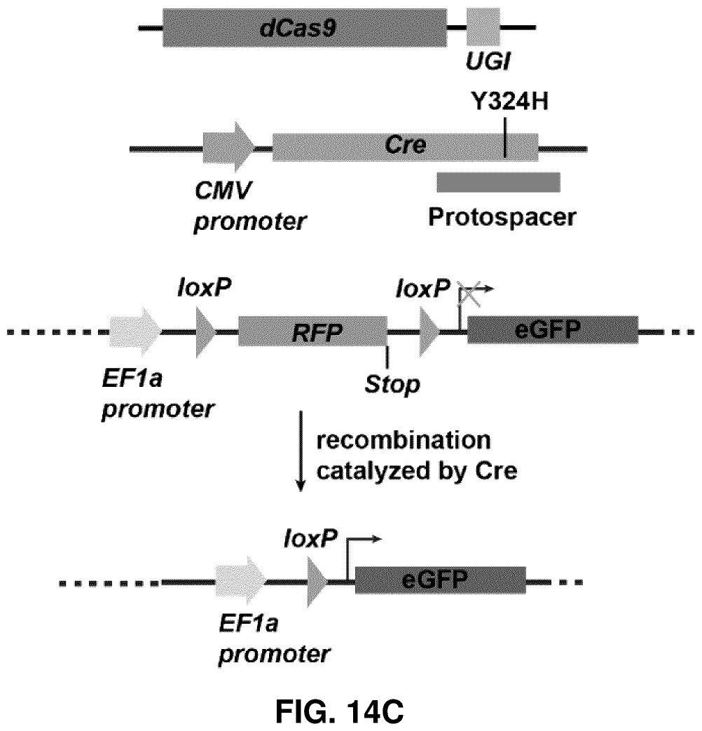

The present disclosure provides aptamers that recruit endogenous effectors for CRISPR-mediated genomic and epigenomic editing. The first endogenous effector targeted by the disclosed intracellular aptamer selection was transcriptional activator c-Myc. As a core regulator of gene expression, c-Myc can act on a wide range of genes, making it a good candidate for multiplexing. This protein has also been implicated in several human cancers and is considered as an “undruggable” target by small molecules or biologics, making it an attractive target for intracellular aptamer development. The RNA library was first pre-enriched by in vitro SELEX until exhibiting a strong bulk affinity to c-Myc, which then was cloned into the tetraloop region of the sgRNA. The plasmid containing the sgRNA-RNA library chimera was packaged into AAV2 vectors and delivered into HEK293T cells such that no more than one virion per cell was delivered. The other plasmids encoding the dSaCas9, the adenoviral helper genes, and the reporter construct (FIG. 14A) were delivered through plasmid transfection. Cells expressing robust fluorescence were enriched by FACS sorting. In the initial rounds of intracellular selection, cells overexpressing c-Myc were used to improve the signal intensity.

The ability to recruit different effectors to allow distinct types of regulation to be executed at different loci greatly expanded the potential of multiplexing. To this end, aptamers were evolved intracellularly to enable the recruitment of endogenous epigenetic remodeling protein Tet1. For the Tet1 aptamer selection, a previously developed methylation reporter construct was used; the construct consisted of a synthetic methylation-sensing promoter Snrpn that controls the expression of eGFP (FIG. 14B). Demethylation of CpG in this promoter was shown to faithfully turn on the reporter gene it controls.

It was predicted that the intracellularly evolved aptamers targeting DNA repair enzyme cytidine deaminase would enable a powerful approach for CRISPR-mediated nucleobase editing. To prevent reversal of the base editing by cell's endogenous repair mechanism, in the cytidine deaminase aptamer selection, an extra 83-amino acid protein uracil DNA glycosylase inhibitor (UGI) that can inhibit base-excision repair was fused to dSaCas9 (FIG. 14C). The addition of the gene encoding UGI only modestly increases the size of the dSaCas9 gene by ˜350 bases (linker included), and was not expected to significantly affect its packaging capability into an AAV capsid. The reporter system included two fragments (FIG. 14C): The first fragment consisted of a CMV promoter and the gene encoding an inactive Cre recombinase with a loss-of-function Y324H mutation in the active site caused by a T to C mutation (TAT->CAT). The sgRNA was designed to target a protospacer within the five-base window of the mutated base. The second fragment consisted of an EF1a promoter-controlled eGFP gene, in which a red fluorescent protein (RFP) gene with a stop codon flanked by two IoxP sites was inserted between the promoter and the eGFP gene. This insertion deactivated the eGFP transcription until a functional Cre excised the RFP insertion between the two IoxP sites. The successful C to U (equivalent to T) repair of the Cre gene via cytidine deaminase recruited by the sgRNA-aptamer-SaCas9 complex produced the functional Cre protein, which excised the RFP insert to express eGFP. The ratio of RFP and eGFP fluorescence was used as the readout for the base editing screen by FACS. High-throughput DNA sequencing was used to quantify the fraction of edited base over the course of the intracellular selection.

### Example 4: AAV Packaging

The present disclosure provides non-nuclease CRISPR editors that can be applied to genome-scale multiplexed transcriptional, epigenetic, and post-translational editing for studying the interplay of the genetic elements in causing cancer drug resistance. The non-nuclease CRISPR editors consisting of only dSaCas9 and effector-binding sgRNA-aptamer chimera were optimized to be packaged into a single AAV capsid. AAV-DJ serotype was chosen as the vector due to its demonstrated utility in gene delivery. The titer of AAV was monitored by qPCR and the amplified DNA cargo was extracted and analyzed by agarose gel to confirm the length and composition of the packaged genes.

### Example 5: Multiplexing Studies

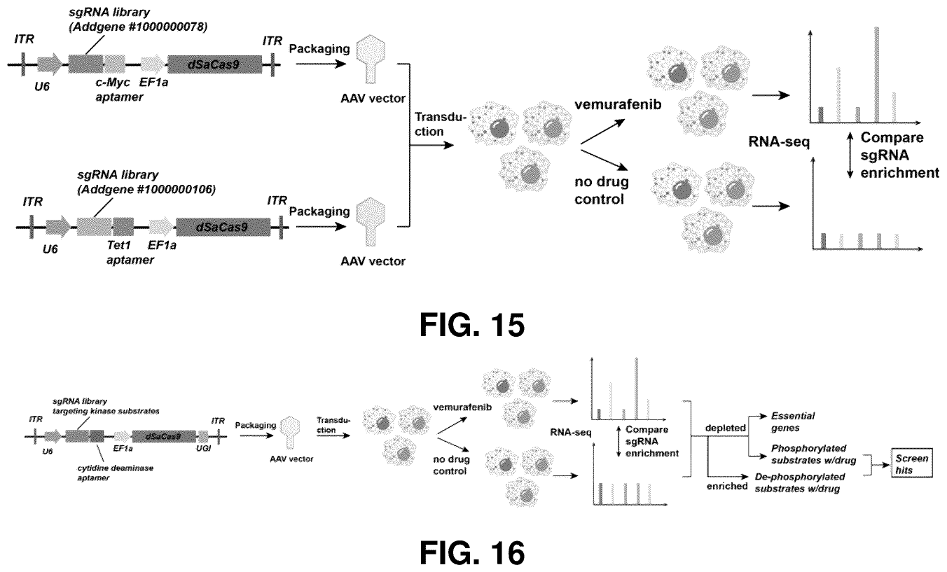

The non-nuclease CRISPR editors were co-delivered and multiplexed to establish a system to model the complex network of multiple genetic and epigenetic factors in the emergence of drug resistance in cancer. A previous study performed genome-scale screening of the protein-coding genes and long non-coding RNA (IncRNA) in A375 melanoma cells that confer resistance to the BRAF inhibitor vemurafenib. However, the interplay between protein-coding genes and IncRNA, and the role of epigenetic modulation in IncRNA transcription in the genome scale still remain elusive. A librar×library genome-scale screen that combined the CRISPR transcriptional activator and the CRISPR DNA methylation editor was performed to simultaneously activate the transcription of protein-coding genes and modulate the transcription of IncRNA through DNA demethylation in A375 cells (FIG. 15). The sgRNA-aptamer chimera of the CRISPR editors were designed to incorporate sgRNA libraries that enable genome-scale targeting of the promoter regions of protein-coding genes and the IncRNA TSS (Addgene Pooled Library #1000000078 and #1000000106) and couple them with c-Myc and Tet1-recruiting aptamers, respectively, to form sgRNA-aptamer chimera in the disclosed non-nuclease CRISPR editors. The Tet1-mediated demethylation of the IncRNA TSS was implicated in the transcription regulation of IncRNA, and it is believed that targeting the TSS of IncRNA by Tet1 enabled unprecedented genome-scale investigation into the regulatory function of the DNA methylation in IncRNA transcription. Protein-coding genes and IncRNA that confer vemurafenib resistance were expected to result in the enrichment of the sgRNA targeting these genes in the cell population after vemurafenib treatment. RNA-seq was employed to identify these enriched sgRNAs and the corresponding genes and IncRNA in the vemurafenib-treated cells compared with the non-treated control. The hits were further validated by the level of vemurafenib resistance when they were overexpressed individually. Next, the confirmed hits of this multiplexed screen were compared with the hits from the previous activation screens acting on protein-coding genes and IncRNA individually to identify the genetic elements that are involved in the synergistic interplay in conferring drug resistance, and the role of DNA methylation in controlling IncRNA expression.

### Kinase Pathway Reconfiguration

Kinase pathway reconfiguration is another important mechanism that cancer cells exploit to acquire drug resistance. A systematic survey of the kinase proteome (so-called kinome) and their substrates in the resistant cells helped to reveal the critical alternative pathways leading to drug resistance. While traditional kinome knockout screens based on transposon, RNAi, or CRISPR-Cas9 nuclease technologies have discovered potentially druggable oncogenes, their utility in the systematic investigation into compensatory resistance mechanisms in cancer cells is still limited by the complexity of the kinase network. The knockout of essential kinase pathways can also result in excessive alterations of cell function and even lethality. Furthermore, eliminating certain kinases from the proteome can cause the concurrent loss of catalysis-independent functions of these kinases, further complicating the screening outcome.

The disclosed method provides a novel strategy using CRISPR-mediated base editing to screen kinase substrates. Rather than knocking out kinases, the approach disclosed herein systematically mutated the phosphorylation sites of a library of putative substrates of the serine/threonine-specific protein kinases to abolish their ability to perform phosphorylation. CRISPR-mediated base editors were employed to mutate serine to phenylalanine/leucine and threonine to isoleucine/methionine at the phosphorylation sites by editing cytosine into uracil (equivalent to thymine) in the serine/threonine codon (FIG. 16). This approach was well-suited to discover “hidden” kinase cascades exploited by the drug-resistant cancer cells, while causing minimal perturbations to the phosphorylation-independent functions of the kinase substrates. As many kinases in the cascade were also substrates of the upstream kinases, mutations of the phosphorylation sites of the activation domain could permanently inactivate these kinases without the need for knocking them out. Previous mass spectrometry profiling studies on the substrates of serine/threonine-specific protein kinases has been recorded in comprehensive databases such as Phospho.ELM and PhosphositePlus, which was used to establish the library of the putative substrates. Locus-specific sgRNA was designed to target the phosphorylation sites of these putative kinase substrates to effect the C to U (equivalent to T) mutation. It is noteworthy that even after optimization editing efficiencies of the CRISPR-mediated base editing was expected to be context-dependent and less than 100%. Therefore, the phosphorylation inhibition by CRISPR-mediated base editing was equivalent to a knockdown rather than a knockout. This property was beneficial when the essential kinase pathways were involved, as the complete knockout of the pathway is believed to cause significant cellular stress. The genes encoding dSaCas9 and the chimeric RNA construct consisting of the sgRNA and the cytidine deaminase aptamer were packaged into an AAV vector for intracellular delivery (FIGS. 14A-14C). Half of the A375 cells edited by the CRISPR base editors were exposed to vemurafenib, and the enrichment and the depletion of the sgRNAs relative to the other half of non-treated cells was analyzed by RNA-seq. The depleted sgRNAs were expected to target genes that belong to two categories: (1) essential genes whose inactivation may lead to cell death or proliferation arrest (e.g., mTOR 58) and (2) genes that are activated by phosphorylation to confer vemurafenib resistance. In contrast, the enriched sgRNAs were likely associated with the kinase substrates that became de-phosphorylated in the resistant cells (FIG. 16). Genes targeted by both the enriched sgRNA and the second category of the depleted sgRNA were further compared with the known mechanisms of the vemurafenib resistance. The genes not covered by the prior reports were considered as candidates for novel targets whose phosphorylation or dephosphorylation may lead to drug resistance and were individually validated knockout and/or overexpression experiments.

Compared to the existing CRISPR systems, the present disclosure provides a novel intracellular selection system for RNA aptamer targeting endogenous effectors, integrates evolved aptamers with the sgRNA to generate modular and multiplexable genomic editors, and enables multiplexed CRISPR-mediated genomic editing simultaneously at transcriptional, epigenetic, and post-translational levels, allows efficient packaging of CRISPR editors into a single AAV capsid. The technology disclosed herein can also be used to investigate the interplay of genes and drugs in the complex gene network that confers drug resistance in cancer cells. The technology of disclosed herein can also be applied to study the complex gene networks in disease and identify treatment options.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Anderson, J. iGEM Depository of Synthetic Promoters. http://parts.igem.org/Part: BBa_J23117 iGEM2006_Berkeley
2. Baugh, C. et al. 2.8 Å Crystal structure of the malachite green aptamer. J. Mol. Biol. 301, 117-128 (2000).
3. Beauchamp, R. L. et al. A high-throughput kinome screen reveals serum/glucocorticoid-regulated kinase 1 as a therapeutic target for NF2-deficient meningiomas. Oncotarget 6, 16981-16997 (2015).
4. Berens, C. et al. A tetracycline-binding RNA aptamer. Bioorganic Med. Chem. 9, 2549-2556 (2001).
5. Bikard, D. et al. Programmable repression and activation of bacterial gene expression using an engineered CRISPR-Cas system. Nucleic Acids Res. 41, 7429-7437 (2013).
6. Buskirk, A. R. et al. In vivo evolution of an RNA-based transcriptional activator. Chem Biol 10, 533-540 (2003).
7. Charpentier, E. et al., Methods and compositions for RNA-directed target DNA modification and for RNA-directed modulation of transcription, AU 2013266968, Jun. 29, 2017.
8. Chen, B. et al. Dynamic imaging of genomic loci in living human cells by an optimized CRISPR/Cas system. Cell 155, 1479-1491 (2013).
9. Chen, J. S. et al. The chemistry of Cas9 and its CRISPR colleagues. Nat. Rev. Chem. 1, 78 (2017).
10. Cong, L. et al. Multiplex Genome Engineering Using CRISPR/Cas Systems. Science 339, 1088-1093 (2013).
11. Dang, C. V. et al. Drugging the ‘undruggable’ cancer targets. Nat. Rev. Cancer 17, 502-508 (2017).
12. Dang, C. V. et al. The c-Myc target gene network. Semin. Cancer Biol. 16, 253-264 (2006).
13. Dominguez, A. A. et al. Beyond editing: repurposing CRISPR-Cas9 for precision genome regulation and interrogation. Nat. Rev. Mol. Cell Biol. 17, 5-15 (2016).
14. Dong, C. et al. Synthetic CRISPR-Cas gene activators for transcriptional reprogramming in bacteria. Nat. Commun. 9, 2489 (2018).
15. Ellington, A. D. et al. In vitro selection of RNA molecules that bind specific ligands. Nature 346, 818 (1990).
16. Erickson, S. B. et al. Precise Photoremovable Perturbation of a Virus-Host Interaction. Angew. Chem. Int. Ed. 56, 4234-4237 (2017).
17. Galonska, C. et al. Genome-wide tracking of dCas9-methyltransferase footprints. Nat. Commun. 9, 597 (2018).
18. Gangloff, Y. et al. Disruption of the Mouse mTOR Gene Leads to Early Postimplantation Lethality and Prohibits Embryonic Stem Cell Development Disruption of the Mouse mTOR Gene Leads to Early Postimplantation Lethality and Prohibits Embryonic Stem Cell Development. Mol. Cell. Biol. 24, 9508-9516 (2004).
19. Gibb, B. et al. Requirements for catalysis in the Cre recombinase active site. Nucleic Acids Res. 38, 5817-5832 (2010).
20. Gilbert, L. A. et al. Genome-Scale CRISPR-Mediated Control of Gene Repression and Activation. Cell 159, 647-661 (2014).
21. Hilton, I. B. et al. Epigenome editing by a CRISPR-Cas9-based acetyltransferase activates genes from promoters and enhancers. Nat. Biotechnol. 33, 510-517 (2015).
22. Hsu, P. D. et al. Development and applications of CRISPR-Cas9 for genome engineering. Cell 157, 1262-1278 (2014).
23. Jiang, F. et al. A Cas9-guide RNA complex preorganized for target DNA recognition. Science 348, 1477-1482 (2015).
24. Jinek, M. et al. A Programmable Dual-RNA-Guided DNA Endonuclease in Adaptive Bacterial Immunity. Science 337, 816-822 (2012).
25. Joung, J. et al. Genome-scale activation screen identifies a IncRNA locus regulating a gene neighbourhood. Nature 548, 343-346 (2017).
26. Kearns, N. A. et al. Functional annotation of native enhancers with a Cas9-histone demethylase fusion. Nat. Methods 12, 401-403 (2015).
27. Kelemen, R. E. et al. A Precise Chemical Strategy To Alter the Receptor Specificity of the Adeno-Associated Virus. Angew. Chem. Int. Ed. 55, 10645-10649 (2016).
28. Kelemen, R. E. et al. Virus-assisted directed evolution of enhanced suppressor tRNAs in mammalian cells. (2018).
29. Kodama, M. et al. In vivo loss-of-function screens identify KPNB1 as a new druggable oncogene in epithelial ovarian cancer. Proc. Natl. Acad. Sci. 114, E7301-E7310 (2017).
30. Komor, A. C. et al. CRISPR-based technologies for the manipulation of eukaryotic genomes. Cell 168, 20-36 (2017).
31. Komor, A. C. et al. Programmable editing of a target base in genomic DNA without double-stranded DNA cleavage. Nature 533, 420-424 (2016).
32. Komor, A. C. et al. Programmable editing of a target base in genomic DNA without double-stranded DNA cleavage. Nature 533, 420-424 (2016).
33. Konermann, S. et al. Genome-scale transcriptional activation by an engineered CRISPR-Cas9 complex. Nature 517, 583-8 (2014).
34. Konermann, S. et al. Systems, methods and compositions for sequence manipulation with optimized functional CRISPR-Cas systems, US 2016/0355797, Dec. 8, 2016.
35. Kosicki, M. et al. Repair of CRISPR-Cas9-induced double-stranded breaks leads to large deletions and complex rearrangements. Nat. Biotechnol. Advanced Publication, doi: 10.1038/nbt.4192 (2018).
36. Kung, J. E. et al. Structural Basis for the Non-catalytic Functions of Protein Kinases. Structure 24, 7-24 (2016).
37. Lau, C.-H. et al. In vivo genome editing in animals using AAV-CRISPR system: applications to translational research of human disease. F1000Research 6, 2153 (2017).
38. Li, J. et al. DNA methylation signature of long noncoding RNA genes during human pre-implantation embryonic development. Oncotarget 8, 56829-56838 (2017).
39. Liao, H. K. et al. In Vivo Target Gene Activation via CRISPR/Cas9-Mediated Trans-epigenetic Modulation. Cell 171, 1495-1507.e15 (2017).
40. Liu, D. R. et al., Extended DNA-sensing gRNAs, U.S. Pat. No. 9,340,800, May 17, 2016.
41. Liu, X. S. et al. Editing DNA Methylation in the Mammalian Genome. Cell 167, 233-247.e17 (2016).
42. Ma, D. et al. Rational Design of Mini-Cas9 for Transcriptional Activation. ACS Synth. Biol. 7, 978-985 (2018).
43. Maeder, M. L. et al. CRISPR RNA-guided activation of endogenous human genes. Nat. Methods 10, 977-979 (2013).
44. Mali, P. et al. CAS9 transcriptional activators for target specificity screening and paired nickases for cooperative genome engineering. Nat. Biotechnol. 31, 833-838 (2013).
45. Mali, P. et al. RNA-Guided Human Genome Engineering via Cas9. Science 823, 823-827 (2013).
46. McCubrey, J. A. et al. Roles of the Raf/MEK/ERK pathway in cell growth, malignant transformation and drug resistance. Biochim. Biophys. Acta-Mol. Cell Res. 1773, 1263-1284 (2007).
47. Mout, R. et al. In Vivo Delivery of CRISPR/Cas9 for Therapeutic Gene Editing: Progress and Challenges. Bioconjug. Chem. 28, 880-884 (2017).
48. Nishida, K. et al. Targeted nucleotide editing using hybrid prokaryotic and vertebrate adaptive immune systems. Science 353, (2016).
49. Nussinov, R. et al. A New View of Pathway-Driven Drug Resistance in Tumor Proliferation. Trends Pharmacol. Sci. 38, 427-437 (2017).
50. Paroo, Z. et al. Challenges for RNAi in vivo. Trends Biotechnol. 22, 390-394 (2004).
51. Pavan, S. et al. A kinome-wide high-content siRNA screen identifies MEK5-ERK5 signaling as critical for breast cancer cell EMT and metastasis. Oncogene 37, 4197-4213 (2018).
52. Pineda, M. et al. Engineered CRISPR Systems for Next Generation Gene Therapies. ACS Synth. Biol. 6, 1614-1626 (2017).
53. Ran, F. A. et al. In vivo genome editing using Staphylococcus aureus Cas9. Nature 520, 186-191 (2015).
54. Rauch, J. et al. The secret life of kinases: Functions beyond catalysis. Cell Commun. Signal. 9, 23 (2011).
55. Rinn, J. L. et al. Methods of making and using guide RNA for use with Cas9 systems, WO 2016/183,402, Nov. 17, 2016.
56. Sedlyarova, N. et al. Natural RNA Polymerase Aptamers Regulate Transcription in E. coli. Mol. Cell 67, 30-43.e6 (2017).
57. Shalem, O. et al. Genome-scale CRISR-Cas9 knockout screening in human cells. Science 343, 84-88 (2014).
58. Shechner, D. M. et al. Multiplexable, locus-specific targeting of long RNAs with CRISPR-Display. Nat. Methods 12, 664-670 (2015).
59. Sredni, S. T. et al. A functional screening of the kinome identifies the Polo-like kinase 4 as a potential therapeutic target for malignant rhabdoid tumors, and possibly, other embryonal tumors of the brain. Pediatr. Blood Cancer 64, 1-10 (2017).
60. Tuerk, C. et al. Systematic evolution of ligands by exponential enrichment: RNA ligands to bacteriophage T4 DNA polymerase. Science 249, 505-510 (1990).
61. Vora, S. et al. Rational design of a compact CRISPR-Cas9 activator for AAV-mediated delivery. bioRxiv 9, 298620 (2018).
62. Zalatan, J. G. et al. Engineering complex synthetic transcriptional programs with CRISPR RNA scaffolds. Cell 160, 339-350 (2015).
63. Zhang, F. et al., Functional screening with optimized functional CRISPR-Cas systems, US 2018/0057810, Mar. 1, 2018.

What is claimed is:

1. A method for an intracellular selection of a CRISPR-associated aptamer (CAP) that interacts with an intracellular target protein, the method comprising:
a) constructing a plurality of CRISPR guide RNAs comprising a randomized library of CAP sequences fused with the one spacer sequence identified in an optimization pre-selection process, wherein the plurality of CRISPR guide RNAs are configured to hybridize to one sequence identified in the optimization process within from 50 to 1000 bases upstream of a transcription start site for a reporter gene comprised on a reporter construct used in the optimization process;
b) constructing a plurality of CRISPR editors, wherein the CRISPR editors comprise the plurality of CRISPR guide RNAs of (a), the reporter construct, and a nuclease-dead Cas9 protein;
c) introducing the CRISPR editors of (b) in host cells, wherein one host cell comprises no more than one CAP sequence;
d) intracellularly expressing the target protein in the host cell; and
e) intracellularly selecting the host cell that comprises a functional CAP from the randomized CAP library that binds to the target protein resulting in change in expression and/or activity of the reporter construct.

2. The method of claim 1 further comprising repeating steps (a), (b), (c), (d), and (e) one or more times.

3. The method of claim 1, wherein the optimization pre-selection process comprises the following steps:
a) constructing a plurality of CRISPR guide RNAs comprising a known aptamer sequence fused with a plurality of spacer sequences, wherein the plurality of spacer sequences is configured to hybridize to a plurality of DNA sequences within from 50 to 1000 bases upstream of a transcription start site for a reporter gene comprised on a reporter construct;
b) introducing CRISPR editors comprising the CRISPR guide RNAs of (a) with the reporter construct and a nuclease-dead Cas9 protein in a host cell;
c) intracellularly expressing a target protein of the known aptamer sequence of (a) in the host cell; and
d) analyzing the host cell for changes in expression and/or activity of the reporter construct and identifying one CRISPR guide RNA comprising the known aptamer sequence in (a) and one spacer sequence which can lead to 1) successful binding between the aptamer and the target protein, 2) successful hybridization of the CRISPR guide RNA and its binding DNA sequence, and 3) optimal distance from the transcription start site resulting in the highest reporter expression and/or activity.

4. The method of claim 1, wherein the nuclease-dead Cas9 protein comprises a miniaturized, catalytically-inactive Cas9 protein comprising SEQ ID NO. 10.

5. The method of claim 1, wherein the intracellular target protein is selected from the group consisting of an RNA polymerase enzyme or subunit, a transcriptional activator, a transcriptional repressor, an epigenetic regulator, an O-GlcNAc transferase, an O-GlcNAcase, an E3 ubiquitin ligase, p53, or a DNA repair enzyme, and wherein the target protein is endogenous to mammalian cells.

6. A method of effecting a genomic or proteomic modification of a host cell using the CAP selected from the method of claim 1, wherein the selected CAP is incorporated into multiplexing CRISPR editors that comprise different spacer sequences that are capable of hybridizing to different genomic sequences.

7. The method of claim 6, wherein each CRISPR editor is packaged in a single adeno-associated virus capsid comprising an AAV2 serotype capsid or an AAV-DJ serotype capsid, and/or a size-sensitive vector.

8. The method of claim 6, wherein the CRISPR editor induces low immunogenicity in the host cell.

9. The method of claim 6, wherein the genomic modification comprises activating transcription of a gene, repressing transcription of a gene, demethylation of DNA, methylation of DNA, RNA cleavage, DNA cleavage, deaminating one or more nucleotide residues, or a combination thereof.

10. The method of claim 6, wherein the protein modification comprises glycosylation, deglycosylation, ubiquitination, or a combination thereof.

11. A method for developing a CRISPR-hybrid system for intracellularly selecting CRISPR guide RNA-fused CAP comprising:
a) designing and validating a CRISPR-hybrid system that consists of four components: a nuclease-dead Cas9 protein, a CRISPR guide RNA-fused CAP library, a target protein-transcriptional activator fusion protein, and a gene encoding a selection marker or a fluorescent reporter;
b) providing at least two intracellular selections of CAP for intracellularly target proteins; and
c) recruiting orthogonal RNA-protein interactions in bacterial and/or mammalian cells.

12. The method of claim 11, wherein the nuclease-dead Cas9 protein comprises a miniaturized, catalytically-inactive Cas9 protein comprising SEQ ID NO. 10.

13. The method of claim 11, wherein the CAP is capable of targeting endogenous proteins.

14. The method of claim 13, wherein the intracellular target protein is selected from the group consisting of an RNA polymerase enzyme or subunit, a transcriptional activator, a transcriptional repressor, an epigenetic regulator, an O-GlcNAc transferase, an O-GlcNAcase, an E3 ubiquitin ligase, p53, and a DNA repair enzyme.

15. The method of claim 11, wherein fluorescent-activated cell-sorting (FACS) is implemented to simultaneously isolate cell populations carrying the functional CAP from unbound species.

16. The method of claim 11, wherein DNA elements encoding the CAP can be delivered by adeno-associated virus (AAV) and/or a size-sensitive vector.

17. An intracellular directed evolution platform for intracellularly selecting CAP against intracellular target proteins, said intracellular directed evolution platform comprises a CRISPR-hybrid system that consists of four components: a nuclease-dead Cas9 protein, a CRISPR guide RNA-fused CAP library, a target protein-transcriptional activator fusion protein, and a gene encoding a selection marker or a fluorescent reporter.

18. The intracellular directed evolution platform of claim 17, wherein the CAP is fused to a CRISPR guide RNA to recruit intracellular target protein to CRISPR-bound genomic sites, and wherein an expression of CRISPR guide RNA containing different CAPs permits simultaneous multiplexed and multifunctional gene regulations.

19. The intracellular directed evolution platform of claim 17, wherein the CRISPR-hybrid system is coupled with fluorescent-activated cell-sorting (FACS) and identify CAP orthogonal to existing aptamer-target protein pairs.

20. The intracellular directed evolution platform of claim 17, wherein an application of orthogonal CAP-target protein pairs in multiplexed CRISPR allows effective simultaneous transcriptional activation and repression of endogenous genes in bacterial and/or mammalian cells.
